(19) United States
(12) Patent Application Publication
(10) Pub. No.: US 2010/0266654 A1
(43) Pub. Date: Oct. 21, 2010

(54) MEDICAL COMPOSITION INCLUDING AN EXTRACELLULAR MATRIX PARTICULATE

(76) Inventors: Jason P. Hodde, West Lafayette, IN (US); Umesh H. Patel, West Lafayette, IN (US)

Correspondence Address: Woodard, Emhardt, Moriarty, McNett & Henry LLP, 111 Monument Circle, Suite 3700, Indianapolis, IN, 46204-5137, US

(21) Appl. No.: 12/824,503

(22) Filed: Jun. 28, 2010

Related U.S. Application Data
(63) Continuation of application No. PCT/US2008/088406, filed on Dec. 29, 2008.
(60) Provisional application No. 61/017,373, filed on Dec. 28, 2007.

(51) Int. Cl. A61K 9/14; A61K 38/00; A61K 31/7088; A61K 33/38; A61K 38/18; A61P 17/02; A61P 31/00

(52) U.S. Cl. 424/405; 424/484; 514/1.1; 514/44 R; 424/130.1; 424/618; 514/9.1

(57) ABSTRACT

Described are medical compositions including a collagenous ECM particulate dispersed in a carrier. Such medical compositions can be applied to at least a portion of a surface of a sheet of collagenous ECM material to form a medical product. Medical compositions and products as described herein find particular use in wound repair. Related methods of manufacture and use are also described.

### REFERENCE TO RELATED APPLICATIONS

The present application is a continuation of International Patent Application No. PCT/US2008/088406, filed Dec. 29, 2008, pending, which claims the benefit of U.S. Provisional Patent Application No. 61/017,373, filed Dec. 28, 2007, expired, both entitled MEDICAL COMPOSITION INCLUDING AN EXTRACELLULAR MATRIX LYOPHILATE, which are hereby incorporated herein by reference in their entirety.

### BACKGROUND

The present invention resides generally in the field of medical compositions and in particular aspects to medical compositions useful, for example, in healing wounds.

As further background, a variety of extracellular matrix (ECM) materials have been proposed for use in medical grafting, cell culture, and other related applications. For instance, medical grafts and cell culture materials containing submucosa derived from small intestine, stomach or urinary bladder tissues, have been proposed. See, e.g., U.S. Pat. Nos. 4,902,508, 4,956,178, 5,281,422, 5,554,389, 6,099,567 and 6,206,931. Medical materials derived from liver basement membrane have also been proposed, for example in U.S. Pat. No. 6,379,710. As well, ECM materials derived from amnion (see e.g. U.S. Pat. Nos. 4,361,552 and 6,576,618) and from renal capsule membrane (see International PCT Patent Application No. WO 03/002165 published Jan. 9, 2003) have been proposed for medical and/or cell culture applications. In addition, Cook Biotech Incorporated, West Lafayette, Indiana, currently manufactures a variety of medical products based upon small intestinal submucosa under the trademarks SURGISIS®, STRATASIS® and OASIS®.

In certain applications, medical materials have been formed into fluidized compositions and used in conjunction with a variety of components directed towards treating a particular condition. For example, U.S. Pat. No. 6,206,931 describes forming a fluidized composition from an extracellular matrix material. Such fluidized compositions are typically formed into gels for use as an injectable graft. Similarly, International PCT Application No. WO 05/020847 discloses an ECM material formed as a gel and including a bioactive component, such as FGF-2. This gel material can also include a particulate ECM material, which is suggested to provide additional material that can function to provide bioactivity to the gel and/or serve as scaffolding material for tissue ingrowth.

A need remains for additional medical compositions and products that can be used in a wide variety of medical applications. The present invention provides such medical compositions and products, as well as methods for preparing and using the same.

### SUMMARY

In one aspect, the present invention provides a medical composition including a particulate of a collagenous extracellular matrix (ECM) material dispersed in a carrier. The collagenous ECM particulate includes at least one bioactive factor and can include particles having an average particle size of at least about 50 μm. In preferred embodiments, the ECM particulate comprises submucosa of a warm-blooded vertebrate.

In another aspect, the present invention provides a medical product including a sheet of a collagenous extracellular matrix (ECM) material having a tissue contacting surface and a surface opposing the tissue contacting surface. At least a portion of the tissue contacting surface is coated with a medical composition of the invention. Such a medical product finds particular use in wound treatment. In preferred embodiments, the sheet of ECM material and ECM particulate both comprise submucosa of a warm-blooded vertebrate.

In another aspect, the present invention provides a method for preparing a medical composition. The method includes providing a collagenous extracellular matrix (ECM) particulate and treating the particulate with a digestion solution such that the particulate becomes solubilized in the acidic solution. The solution including the solubilized particulate is dried to provide a treated collagenous ECM particulate. The treated collagenous ECM particulate is dispersed in a carrier to form the medical composition. In preferred embodiments, the carrier is non-collagenous and the ECM particulate comprises submucosa from a warm-blooded vertebrate. Also in preferred embodiments, the solubilized particulate is dried by lyophilization to form the treated collagenous ECM particulate as a lyophilate composition.

In another aspect, the present invention provides a method for treating a patient. The method includes providing a medical composition of the invention and applying the composition to a patient. The medical composition is applied to an external or internal structure on the patient. In an alternate embodiment, the medical composition can be applied to the tissue contacting surface of a sheet of collagenous ECM material before being applied to a patient. In still another embodiment, a medical composition can be applied to the patient and subsequently covered with a sheet of a collagenous ECM material.

Additional embodiments as well as features and advantages of the invention will be apparent from the further descriptions herein.

### BRIEF DESCRIPTION OF THE DRAWINGS

FIG. 1 is flow diagram illustrating the processing steps for preparing a medical composition for use in a medical product of the invention.

FIG. 2 provides a perspective view of a medical product of the invention containing a sheet of a collagenous extracellular matrix (ECM) material including a medical composition on a surface thereof.

FIG. 3 provides a perspective view of a medical producing of the invention containing multiple sheets of a collagenous extracellular matrix (ECM) material formed as a laminate and including a medical composition on a surface thereof.

### DETAILED DESCRIPTION

As disclosed above, the present invention provides medical compositions and products formed therefrom useful in a wide variety of medical applications. Such medical compositions include a particulate of a collagenous ECM material dispersed in a carrier. The particulate includes particles that can be solubilized in a digestion solution, preferably an acidic aqueous medium, which allows for a more efficient delivery of factors both native and non-native to the ECM material when the composition is applied to a patient.

Medical compositions of the invention can be used to treat a variety of medical conditions, including wounds such as partial or full thickness topical wounds affecting dermal tissue. In one embodiment, a medical composition of the invention can be directly applied to a wound, and can be reapplied as needed. Alternatively, a wound can be first contacted with a medical composition as described herein and a sheet of a collagenous ECM material can be placed over the medical composition. Such an embodiment is particularly useful in treating deep wounds in a patient. In another embodiment, a surface of a sheet of collagenous ECM material can be coated, at least in part, with a medical composition and subsequently applied to a patient as a wound dressing. In preferred embodiments, a substantial portion of the sheet of ECM material is coated with a medical composition. The advantage of using a collagenous ECM material in the medical compositions and products of the invention is that they can elute substances contained therein or thereon locally; thereby diminishing the need for systemic administration of the substance and minimizing the risk of systemic toxicity and adverse reactions associated with injectable products containing these substances.

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated medical products, and further applications of the principles of the invention as illustrated therein are herein contemplated as would normally occur to one skilled in the art to which the invention relates.

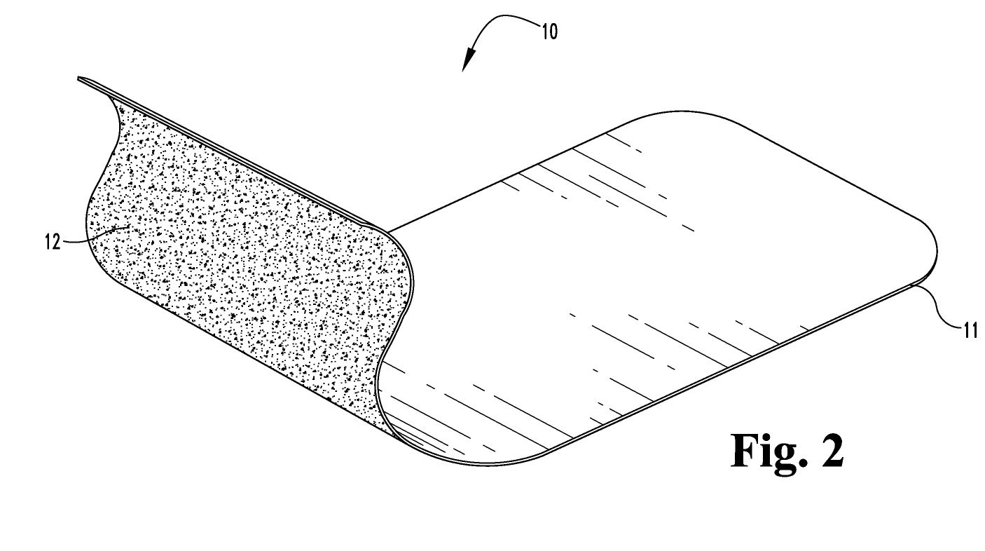

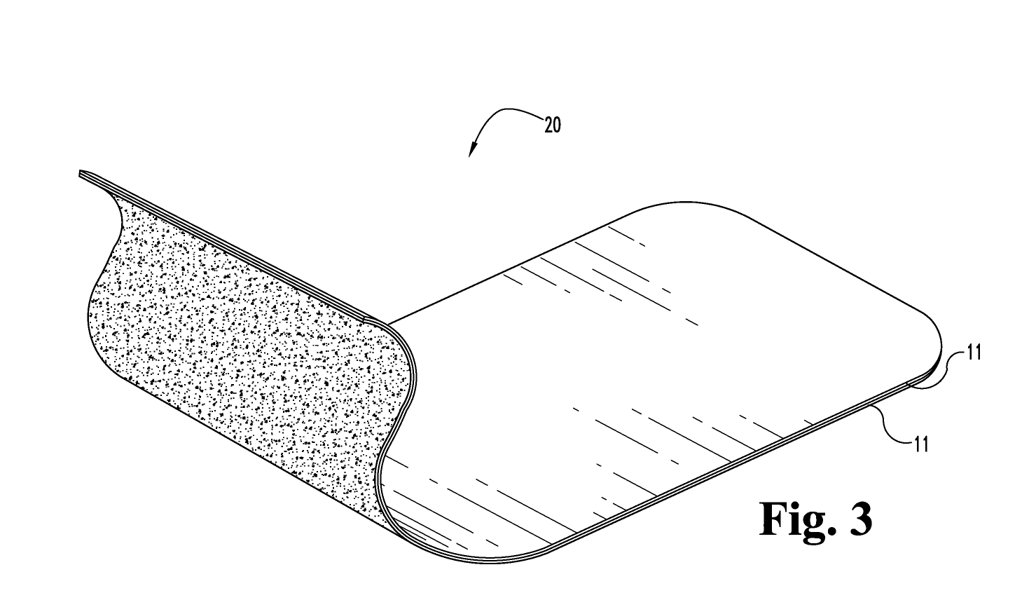

With reference now to FIG. 2, shown is a medical product 10 of the invention. The medical product includes a sheet of a collagenous ECM material 11 having a tissue contacting surface and a surface opposing the tissue contacting surface. The tissue contacting surface includes a coating of a medical composition 12 including a collagenous ECM particulate dispersed in a carrier. FIG. 3 illustrates a similar medical product 20 formed of multiple sheets of a collagenous ECM material. The multiple sheets of a collagenous ECM material are bonded together to form a laminate including a tissue contacting surface and a surface opposing the tissue contacting surface. The tissue contacting surface includes a coating of a medical composition including a collagenous ECM particulate dispersed in a carrier.

Although not pictured, a medical product of the invention, such as medical products 10 and 20, can include a release paper covering the surface of the sheet of collagenous ECM material coated with a medical composition. Such a release paper finds use, for instance, to protect the medical composition prior to its application to tissue or device. In particular, a release paper can be included where a medical product is contained within a sterile package prior to use. A release paper can be made of any suitable material and is preferably made of a non-stick material, such as Tyvek®. This material can stick to the medical composition, but is generally non-adhesive towards other surfaces. In this respect, a non-adhesive release paper will not stick to the walls of a sterile package when the medical product is stored for any period of time. Just prior to use, the release paper can be removed, and the medical product can be applied to a desired tissue.

Generally, when a sheet of a collagenous ECM material is contemplated for use in the invention, the sheet of material is cut or otherwise configured to a desired size for its end use. The sheet of material is preferably sized larger than the tissue defect to which it is applied. Sizing the sheet of material in this way allows for easy attachment to the surrounding tissue.

Although the medical composition can be sufficient to secure the medical product in place, it may, in certain instances, be advantageous to more securely attach the medical product to tissue or other structure. For example, once the sized medical product has been placed on, in, or around the area in need of treatment, the medical product can be more securely attached to the surrounding tissue or other structure using any of several known suitable attachment means. Suitable attachment means include, for example, stapling, suturing, and the like. Preferably, the medical material is more securely attached to the surrounding tissue or other structure by sutures. There are a variety of synthetic materials currently available in the art for use as sutures. For example, sutures comprising Prolene™, Vicryl®, Mersilene™, Panacryl®, and Monocryl™, are contemplated for use in the invention. Other suture materials will be well known to those skilled in the art. Medical adhesives as generally known in the art can also be used in conjunction with the medical products of the invention.

A medical product of the invention can be in a dehydrated or hydrated state. Dehydration of a medical product of the invention can be achieved by any means known in the art. Preferably, dehydration is accomplished by either air drying, lyophilization or vacuum pressure and can simultaneously be utilized to bond the layer of medical material and medical composition together. Alternatively, the medical product can be in a hydrated state. Typically, a medical product will be dehydrated when it is to be stored for a period of time. Any suitable solution can then be used to rehydrate the medical product prior to use. Preferably, the rehydration solution comprises water or buffered saline. In certain embodiments, hydrating the medical product will activate the adhesive, if contained thereon, such that the medical product can adhere to tissue or a device. The above-described methods of dehydration and rehydration of the medical product allow for an effective shelf life and convenient packaging.

In certain embodiments, the medical product can be crosslinked. A medical product can be crosslinked once formed, or the sheet(s) of a collagenous ECM material can be crosslinked prior to applying the medical composition to the material, or both. Increasing the amount (or number) of crosslinkages within the medical product or between two or more layers of ECM material can be used to enhance its strength. However, crosslinkages within the ECM material or medical product may also affect its remodelability. Consequently, in certain embodiments, the ECM material or medical product will substantially retain its native level of crosslinking, or the amount of added crosslinkages within the ECM material or medical product can be judiciously selected depending upon the desired treatment regime. In many cases, the ECM material or medical product will exhibit remodelable properties such that the remodeling process occurs over the course of several days or several weeks. In preferred embodiments, the remodeling process occurs within a matter of about 5 days to about 12 weeks.

The medical products of the invention can be provided in sterile packaging suitable for medical products. Sterilization may be achieved, for example, by irradiation, ethylene oxide gas, or any other suitable sterilization technique, and the materials and other properties of the medical packaging will be selected accordingly.

For use in the present invention, introduced crosslinking of the medical product, especially any sheet-form ECM component of the product, may be achieved by photo-crosslinking techniques, or by the application of a crosslinking agent, such as by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

When multiple sheets of a collagenous ECM material are used to form a laminate material, the layers of the laminate can be additionally crosslinked to bond multiple sheets of a collagenous ECM material to one another. Thus, additional crosslinking may be added to individual layers prior to coupling to one another, during coupling to one another, and/or after coupling to one another.

It is advantageous to use a remodelable material for use in the medical compositions, products and methods of the present invention, and particular advantage can be provided by including a remodelable collagenous material. Such remodelable collagenous materials can be provided, for example, by collagenous materials isolated from a suitable tissue source from a warm-blooded vertebrate, and especially a mammal. Reconstituted or naturally-derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

The submucosa can be derived from any suitable organ or other biological structure, including for example submucosa derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source. For additional information as to submucosa useful in the present invention, and its isolation and treatment, reference can be made, for example, to U.S. Pat. Nos. 4,902,508, 5,554,389, 5,993,844, 6,206,931, and 6,099,567.

As prepared, the submucosa material and any other ECM used (including any used in sheet form and/or that reduced to a particulate) may optionally retain growth factors or other bioactive components native to the source tissue. For example, the submucosa or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with a device including the material. In this regard, angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, anti-inflammatory agents, proteins or peptide fragments thereof, genes or nucleic acid fragments thereof, growth factors, analgesic agents, antibodies or immunologically active fragments thereof, thrombus-promoting substances such as blood clotting factors, e.g. thrombin, fibrinogen, and the like. A non-native bioactive component can be included in the medical composition. For example, a non-native bioactive component can be mixed with the carrier prior to, in conjunction with or after the ECM particulate is added or, alternatively, a non-native bioactive component can be added to the medical composition after it is formed. In embodiments where a medical product is contemplated, these substances may be applied to a sheet of a collagenous ECM material as a premanufactured step, immediately prior to the procedure (e.g. by soaking the material in a solution containing a suitable antibiotic such as cefazolin), or during or after engraftment of the medical product in the patient.

A non-native bioactive component can be applied to a submucosa or other ECM tissue by any suitable means. Suitable means include, for example, mixing, spraying, impregnating, dipping, etc. Similarly, if other chemical or biological components are included in the ECM tissue, the non-native bioactive component can be applied either before, in conjunction with, or after these other components.

The inventive composite ECMs can also serve as a collagenous matrix in compositions for producing transformed cells. The techniques for cell transformation have been described in International Publication Nos. WO 96/25179 and WO 95/22611; the disclosures of which are expressly incorporated herein by reference. Preferably, purified composite ECMs of the present invention, for example in fluidized or paste form, is included in the cell transformation compositions, in combination with a recombinant vector (e.g. a plasmid) containing a nucleic acid sequence with which in vitro or in vivo target cells are to be genetically transformed.

Still further, the inventive methods herein may use a biomaterial that serves as a matrix that can support and produce genetically modified cells, (see, e.g., International Publication No. WO 96/25179 dated 22 Aug. 1996, publishing International Application No. PCT/US96/02136 filed 16 Feb. 1996; and International Publication No. WO 95/22611 dated 24 Aug. 1995, publishing International Application No. PCT/US95/02251 filed 21 Feb. 1995). Such compositions for genetically modifying cells can include an ECM such as submucosa or another collagenous biomaterial as a three dimensional construct or a fluidized or flowable material in combination with a nucleic acid molecule containing a sequence to be expressed in cells, e.g. a recombinant vector such as a plasmid containing a nucleic acid sequence with which in vitro or in vivo target cells are to be genetically modified.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

In additional embodiments, medical products of the invention can include ECM's or other collagenous materials that have been subjected to processes that expand the materials. In certain forms, such expanded materials can be formed by the controlled contact of an ECM material with one or more alkaline substances until the material expands, and the isolation of the expanded material. Illustratively, the contacting can be sufficient to expand the ECM material to at least 120% of (i.e. 1.2 times) its original bulk volume, or in some forms to at least about two times its original volume. Thereafter, the expanded material can optionally be isolated from the alkaline medium, e.g. by neutralization and/or rinsing. The collected, expanded material can be used in any suitable manner in the preparation of a medical device. Illustratively, the expanded material can be enriched with bioactive components, dried, and/or molded, etc., in the formation of a graft construct of a desired shape or configuration. In certain embodiments, a medical graft material and/or device formed with the expanded ECM material can be highly compressible (or expandable) such that the material can be compressed for delivery, such as from within the lumen of a cannulated delivery device, and thereafter expand upon deployment from the device so as to become anchored within a patient and/or cause closure of a tract within the patient.

Expanded collagenous or ECM materials can be formed by the controlled contact of a collagenous or ECM material with an aqueous solution or other medium containing sodium hydroxide. Alkaline treatment of the material can cause changes in the physical structure of the material that in turn cause it to expand. Such changes may include denaturation of the collagen in the material. In certain embodiments, it is preferred to expand the material to at least about three, at least about four, at least about 5, or at least about 6 or even more times its original bulk volume. The magnitude of the expansion is related to several factors, including for instance the concentration or pH of the alkaline medium, exposure time, and temperature used in the treatment of the material to be expanded.

ECM materials that can be processed to make expanded materials can include any of those disclosed herein or other suitable ECM's. Typical such ECM materials will include a network of collagen fibrils having naturally-occurring intramolecular cross links and naturally-occurring intermolecular cross links. Upon expansion processing as described herein, the naturally-occurring intramolecular cross links and naturally-occurring intermolecular cross links can be retained in the processed collagenous matrix material sufficiently to maintain the collagenous matrix material as an intact collagenous sheet material; however, collagen fibrils in the collagenous sheet material can be denatured, and the collagenous sheet material can have an alkaline-processed thickness that is greater than the thickness of the starting material, for example at least 120% of the original thickness, or at least twice the original thickness.

Illustratively, the concentration of the alkaline substance for treatment of the remodelable material can be in the range of about 0.5 to about 2 M, with a concentration of about 1 M being more preferable. Additionally, the pH of the alkaline substance can in certain embodiments range from about 8 to about 14. In preferred aspects, the alkaline substance will have a pH of from about 10 to about 14, and most preferably of from about 12 to about 14.

In addition to concentration and pH, other factors such as temperature and exposure time will contribute to the extent of expansion, as discussed above. In this respect, in certain variants, the exposure of the collagenous material to the alkaline substance is performed at a temperature of about 4 to about 45° C. In preferred embodiments, the exposure is performed at a temperature of about 25 to about 40° C., with 37° C. being most preferred. Moreover, the exposure time can range from at least about one minute up to about 5 hours or more. In some embodiments, the exposure time is about 1 to about 2 hours. In a particularly preferred embodiment, the collagenous material is exposed to a 1 M solution of NaOH having a pH of 14 at a temperature of about 37° C. for about 1.5 to 2 hours. Such treatment results in collagen denaturation and a substantial expansion of the remodelable material. Denaturation of the collagen matrix of the material can be observed as a change in the collagen packing characteristics of the material, for example a substantial disruption of a tightly bound collagenous network of the starting material. A non-expanded ECM or other collagenous material can have a tightly bound collagenous network presenting a substantially uniform, continuous surface when viewed by the naked eye or under moderate magnification, e.g. 100× magnification. Conversely, an expanded collagenous material can have a surface that is quite different, in that the surface is not continuous but rather presents collagen strands or bundles in many regions that are separated by substantial gaps in material between the strands or bundles when viewed under the same magnification, e.g. about 100×. Consequently, an expanded collagenous material typically appears more porous than a corresponding non-expanded collagenous material. Moreover, in many instances, the expanded collagenous material can be demonstrated as having increased porosity, e.g. by measuring for an increased permeability to water or other fluid passage as compared to the non-treated starting material. The more foamy and porous structure of an expanded ECM or other collagenous material can allow the material to be cast or otherwise prepared into a variety of sponge or foam shapes for use in the preparation of medical materials and devices. It can further allow for the preparation of constructs that are highly compressible and which expand after compression. Such properties can be useful, for example, when the prepared medical graft material is to be compressed and loaded into a deployment device (e.g. a lumen thereof) for delivery into a patient, and thereafter deployed to expand at the implant site.

After such alkaline treatments, the material can be isolated from the alkaline medium and processed for further use. Illustratively, the collected material can be neutralized and/or rinsed with water to remove the alkalinity from the material, prior to further processing of the material to form either an ECM material for application of a medical composition and/or an ECM particulate for use in a medical composition.

A starting ECM material (i.e., prior to treatment with the alkaline substance) can optionally include a variety of bioactive or other non-collagenous components including, for example, growth factors, glycoproteins, glycosaminoglycans, proteoglycans, nucleic acids, and lipids. Treating the material with an alkaline substance may reduce the quantity of one, some or all of such non-collagenous components contained within the material. In certain embodiments, controlled treatment of the remodelable material with an alkaline substance will be sufficient to create a remodelable collagenous material which is substantially devoid of nucleic acids and lipids, and potentially also of growth factors, glycoproteins, glycosaminoglycans, and proteoglycans. This may be true for other processing techniques as discussed herein, such as the controlled treatment of the material with a detergent.

In certain embodiments, one or more bioactive components, exogenous or endogenous, for example, similar to those removed from an ECM material (i.e., both non-expanded and expanded materials) during processing, can be returned to the material. For example, an ECM material can include a collagenous material which has been depleted of nucleic acids and lipids, but which has been replenished with growth factors, glycoproteins, glycosaminoglycans, and/or proteoglycans. These bioactive components can be returned to the material by any suitable method. For instance, in certain forms a tissue extract, such as is discussed in U.S. Pat. No. 6,375,989, containing these components can be prepared and applied to an ECM collagenous material. In one embodiment, the ECM can be incubated in a tissue extract for a sufficient time to allow bioactive components contained therein to associate with the ECM material. The tissue extract may, for example, be obtained from non-expanded collagenous tissue of the same type used to prepare the expanded material. Other means for returning or introducing bioactive components to an ECM material include spraying, impregnating, dipping, etc. as known in the art. By way of example, an ECM material may be modified by the addition of one or more growth factors such as basic fibroblast growth factor (FGF-2), transforming growth factor beta (TGF beta), epidermal growth factor (EGF), platelet derived growth factor (PDGF), and/or cartilage derived growth factor (CDGF). As well, other biological components may be added to an ECM material, such as heparin, heparin sulfate, hyaluronic acid, fibronectin and the like. Thus, generally speaking, an ECM material may include a bioactive component that induces, directly or indirectly, a cellular response such as a change in cell morphology, proliferation, growth, protein or gene expression similar to a non-expanded collagenous material.

In certain embodiments of the invention where a medical product is provided in sheet form, the collagenous ECM material of the medical product will have a thickness in the range of about 50 to about 1000 microns, more preferably about 100 to 600 microns, and most preferably about 100 to about 350 microns. If necessary or desired, a multilaminate medical product can be used. For example, a plurality of (i.e. two or more) layers of a biocompatible material, for example submucosa-containing or other ECM material, can be bonded together to form a multilaminate structure. Illustratively, two, three, four, five, six, seven, or eight or more layers of a biocompatible material can be bonded together to provide a multilaminate bolster material. In certain embodiments, two to six collagenous, submucosa-containing layers isolated from intestinal tissue of a warm-blooded vertebrate, particularly small intestinal tissue, are bonded together to provide the a laminate for use in preparation of a medical product. Porcine-derived small intestinal tissue is preferred for this purpose. The layers of collagenous tissue can be bonded together in any suitable fashion, including dehydrothermal bonding under heated, non-heated or lyophilization conditions, using adhesives as described herein, glues or other bonding agents, crosslinking with chemical agents or radiation (including UV radiation), or any combination of these with each other or other suitable methods.

Turning now to a discussion of the medical composition, said composition can be applied to at least a portion of a tissue contacting surface of a collagenous ECM material or can be applied to tissue and optionally covered by a collagenous ECM material. In embodiments where the medical composition is applied to an ECM material, the application means can include, for example, brushing, spraying, impregnating, dipping, etc. Typically, a substantial portion of a surface of a medical material is coated with the medical composition. By “substantial portion” is meant that at least about 75% of a surface of an ECM material is coated with a medical composition. The medical composition can be applied to the ECM material at the point of use, or in a pre-applied configuration. In certain embodiments, a pre-applied medical composition can be covered with release paper or similar material to protect the composition during shipping and handling. The release paper can then be removed prior to use as described previously.

A medical composition as used herein includes a carrier and a particulate of a collagenous ECM material. The carrier can be any suitable carrier generally known in the art, and is preferably a non-collagenous carrier. Non-collagenous carriers generally include, for example, petroleum or white petrolatum. Collagenous carriers can also be used in the context invention and include, for example, a submucosa gel. In certain preferred embodiments, the carrier will be comprised less than 50% by weight of water, more preferably less than about 20% by weight of water. In some embodiments, the carrier can be substantially free of water, which as used herein means that the carrier contains less than about 10% by weight of water. Such carriers can include semi-solid organic carrier materials such as those mentioned above, fats, oils derived from plants or animals, lipids, organic polyols, or other suitable organic carrier materials, preferably having a viscosity greater than that of water at 25° C. In certain embodiments, the carrier comprises white petrolatum in combination with one or more less viscous organic carrier materials, for example including one, some or all of the following: stearyl alcohol, isopropyl myrisate, sorbitan monooleate, polyoxyl 40 stearate, and propylene glycol. Such carriers can also include a preservative, such as methylparaben, and water.

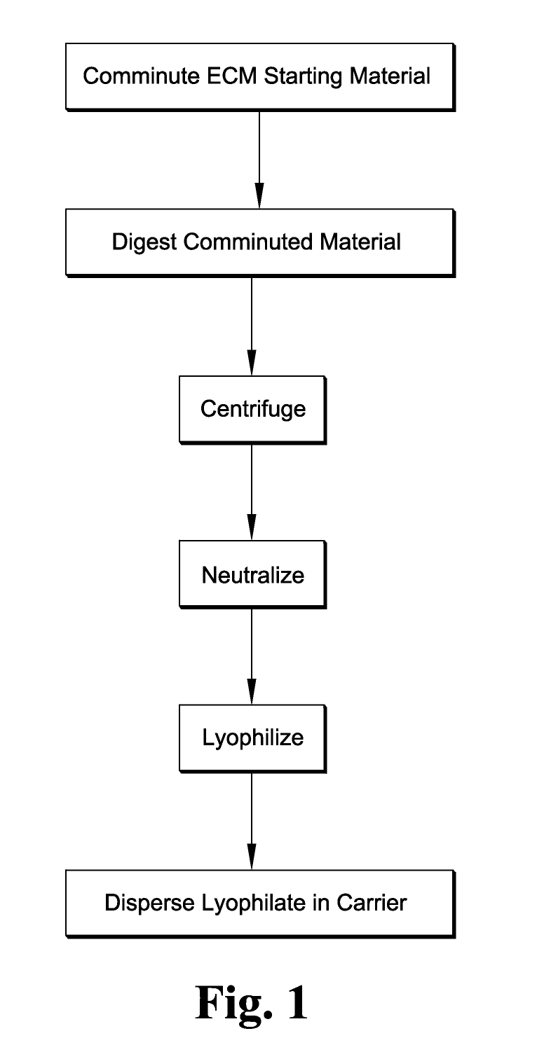

To form a medical composition of the invention as illustrated in FIG. 1, a particulate ECM material is prepared and is subsequently dispersed in a carrier. For example, a particulate ECM material having an average particle size of about 50 microns to about 500 microns may be dispersed in the carrier, more preferably the particulate has an average particle size of about 100 microns to about 400 microns. In certain embodiments, the average particle size for the particulate ECM material will be at least about 75 microns, for example in the range of 75 to 500 microns. The ECM particulate can be added in any suitable amount relative to the carrier, with preferred ECM particulate to carrier weight ratios (based on dry solids) being about 0.1:1 to about 200:1, more preferably in the range of 1:1 to about 100:1. The inclusion of such ECM particulates in the medical composition can serve to provide material that can function to provide bioactivity to the composition (e.g. itself including FGF-2 and/or other growth factors or bioactive substances as discussed herein) and/or serve as scaffolding material for tissue ingrowth. In addition, the particulate includes particles, which are soluble in a digestion solution, preferably an acidic aqueous medium. Such solubilization provides a more efficient delivery of native and non-native bioactive components included n the medical composition.

With respect to the particulate, said particulate is preferably formed by digesting a source of a collagenous ECM material in a digestion solution followed by a drying step to form the particulate. In a particularly preferred embodiment, the particulate is formed by cutting, tearing, grinding or otherwise comminuting a source of a collagenous ECM material as described above to form a particulate. The particulate is digested in a solution, typically an acidic solution, to solubilize the particulate and form a solution containing components of the ECM material. The solution containing the solubilized particulate material, or a suspension including precipitated material formed by neutralizing the solution (e.g. with NaOH or another base), is dried to form a treated collagenous ECM particulate. Preferably, the solubilized particulate material or subsequently precipitated material is dried by lyophilization (forming a particulate lyophilate composition) but can also be accomplished by other means known in the art, such as air-drying or vacuum pressure. Once formed, the particulate can be dispersed in a carrier by any suitable means known in the art, such as mixing, stirring, folding, etc. In preferred embodiments, the dried particulate can retain native bioactive factors naturally present in the source ECM material. Such native bioactive factors can include, for example, one or more native growth factors such as basic fibroblast growth factor (FGF-2), transforming growth factor beta (TGF-beta), epidermal growth factor (EGF), and/or platelet derived growth factor (PDGF). The process of solubilizing the starting, solid ECM material and then processing the solubilized material to a solid particulate again while retaining the non-collagenous components (e.g. native bioactive agents such as growth factors) in the particulate preparation, can modify or enhance the properties of the particulates, including for example the availability of the native bioactive factors in the final dried particulate composition, and the structure or organization of the collagen in the composition. It will be understood that the amount of particulate contained in the medical composition can be varied to achieve the desired level of treatment.

The acidic solution used to digest the source of a collagenous ECM material generally has a molarity or normality of about 0.001-0.1 M or N. In preferred embodiments, the acidic solution includes 0.01 M hydrochloric acid. The acidic solution can further include an enzyme to assist in the digestion of the ECM particulate. Any suitable enzyme known in the art can be used for this purpose. Pepsin is a particularly preferred enzyme. In preferred embodiments, the enzyme is present in an amount of about 0.1-1g/L.

It will be understood from the descriptions herein that other methods of obtaining particulate ECM can be used in other inventive embodiments, including processing ECM sheet materials by simple grinding, shearing (e.g. suspended in water or another liquid, such as in a blender), or other techniques.

Medical compositions of the invention can include bioactive agents that are not native to the ECM material(s) in the composition. As examples, the non-native bioactive component can be an antibiotic, an anti inflammatory agent, a protein or peptide fragment thereof, a gene or nucleic acid fragment thereof, a growth factor, an analgesic agent, or an antibody or immunologically active fragment thereof. Such bioactive agents can be incorporated in the carrier along with the particulate ECM material and/or can be incorporated in or upon any ECM sheet of the product.

In certain preferred embodiments, medical products of the invention including an ECM particulate dispersed in a flowable carrier can also include an antibiotic agent, e.g. suspended or dissolved in the carrier. Suitable antibiotic agents for these purposes include, for example, silver or silver salts such as silver sulfadiazine, amphotericin B, cefoperazone, ciprofloxacin, gentamicin sulfate, neomycin sulfate, hydrochloride neomycin sulfate, nystatin, polymyxin B sulfate, polymixin B sulfate, bacitracin zinc, tpbramycin sulfate, vancomycin hydrochloride, or any combination of some or all of these antibiotic agents.

Medical products of the invention including an ECM particulate dispersed in a flowable carrier can also include nucleic acid molecules, including for example DNA molecules encoding growth factors such as platelet derived growth factor, transforming growth factor beta-1, fibroblast growth factor, and/or vascular endothelial growth factor. The encoded growth factor is preferably a human growth factor. Such nucleic acid molecules can be included as an alternative or in addition to an antibiotic as discussed above, and can be included in a form to be taken up and expressed by cells of the patient at the site of application of the composition (e.g. an injury to dermal tissue such as a topical partial or full thickness wound) to provide a lasting therapeutic effect at the site. Such DNA or other nucleic acid molecules can, for example, be delivered via vectors such as viral (e.g. adenoviral) vectors and/or plasmids.

Medical compositions including the ECM particulate and carrier, and optionally other ingredients such as an antibiotic agent and/or a nucleic acid molecule as discussed above, can in certain embodiments of the invention be applied to or used in conjunction with an ECM sheet material. For example, in the treatment of partial or full thickness topical wounds, an amount of the ECM particulate/carrier mixture composition can be applied to the wound and an ECM sheet material can be positioned over the mixture composition. This can facilitate delivery of therapy to the deeper regions of the wound, which can be difficult to achieve with an ECM sheet material alone. To achieve this arrangement, the ECM particulate/carrier mixture composition can be provided applied to the ECM sheet material, and the combination applied to the wound, or the ECM particulate/carrier mixture composition can be first applied to the wound, followed by an overlying ECM sheet material. These and other modes of application of therapy using products and compositions of the invention will be apparent from the descriptions herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. In addition, all publications cited herein are indicative of the abilities of those of ordinary skill in the art and are hereby incorporated by reference in their entirety as if individually incorporated by reference and fully set forth.

What is claimed is:

1. A medical composition, comprising:
a carrier; and
a particulate of a collagenous ECM material including at least one bioactive factor, said particulate including particles having an average particle size of at least about 50 μm;
wherein said particulate is dispersed in the carrier to form the medical composition.

2. The medical product of claim 1, wherein said carrier comprises a non-collagenous material.

3. The medical product of claim 2, wherein said carrier comprises white petrolatum.

4. The medical product of claim 3, wherein said carrier further comprises stearyl alcohol, isopropyl myrisate, sorbitan monooleate, polyoxyl 40 stearate, propylene glycol, water, and methylparaben.

5. The medical product of claim 1, wherein said medical composition further comprises a non-native bioactive component.

6. The medical product of claim 5, wherein said non-native bioactive component is selected from the group consisting of an antibiotic, an anti inflammatory agent, a protein or peptide fragment thereof, a gene or nucleic acid fragment thereof, a growth factor an analgesic agent, and an antibody or immunologically active fragment thereof.

7. The medical product of any of claims 1, wherein said collagenous ECM material comprises submucosa.

8. The medical product of claim 7, wherein said submucosa is intestinal, urinary bladder or stomach submucosa.

9. The medical product of claim 8, wherein said submucosa is small intestinal submucosa (SIS).

10. A medical product, comprising:
a sheet of a collagenous extracellular matrix (ECM) material including a tissue contacting surface and a surface opposing said tissue contacting surface; and
a medical composition of claim 1;
wherein at least a portion of the tissue contacting surface of the sheet of a collagenous ECM material is coated with the medical composition.

11. A method for treating a patient, comprising:
providing a medical composition of any of claims 1; and
applying said medical composition to the patient.

12. The method of claim 11, wherein the method further comprises applying a sheet of a collagenous extracellular matrix (ECM) material to the patient such that it substantially covers the medical composition.

13. The method of claim 11, wherein said patient is treated for a wound.

14. A method for preparing a medical composition, comprising:
providing a collagenous extracellular matrix (ECM) material particulate;
treating the ECM particulate with a digestion solution to form solubilized ECM components;
reforming ECM particles from the solubilized ECM components to provide a treated collagenous ECM particulate; and
dispersing said treated collagenous ECM particulate in a carrier to form the medical composition.

15. The method of claim 14, wherein said collagenous ECM particulate is provided by freezing a source of ECM material and comminuting said frozen source of collagenous ECM material to form a collagenous ECM particulate.

16. The method of claim 14, wherein said digestion solution comprises hydrochloric acid having a molarity of from about 0.001 to about 0.1 M.

17. The method of claim 14, wherein said digestion solution further comprises an enzyme in an amount of from about 0.1 to about 1 g/L.

18. The method of claim 17, wherein said enzyme comprises pepsin.

19. The method of claim 14, wherein said method further comprises applying the medical composition to at least a portion of a surface of a sheet of a collagenous ECM material so as to form a medical product.

20. The method of claim 19, wherein said medical composition is applied to a substantial portion of a surface of a sheet of a collagenous ECM material.

21. The method of any of claims 14, wherein said collagenous ECM material particulate and sheet of a collagenous ECM material each comprise submucosa.

22. The method of claim 21, wherein said submucosa is intestinal, urinary bladder or stomach submucosa.

23. The method of claim 22, wherein said submucosa is small intestinal submucosa (SIS).

24. The composition of claim 1, also comprising an antibiotic agent.

25. The composition of claim 24, wherein the antibiotic agent is a silver salt.

26. The composition of claim 25, wherein the silver salt is silver sulfadiazine.

27. The composition of claim 26, wherein the carrier comprises white petrolatum.

28. The composition of claim 1, also comprising a nucleic acid molecule encoding a growth factor.

29. The composition of claim 1, wherein the carrier is comprised less than 50% by weight of water.

30. A composition, comprising:
a flowable carrier; and
a particulate collagenous extracellular matrix (ECM) material containing at least one bioactive native growth factor suspended in the carrier, said particulate ECM material prepared by treating a solid collagenous ECM material retaining the at least one bioactive native growth factor with a digestion solution to form solubilized ECM components in the digestion solution, and then forming said particulate collagenous ECM material from said solubilized ECM components.

31. The composition of claim 30, wherein the at least one bioactive native growth factor is fibroblast growth factor-2.
